(19) United States
(12) Patent Application Publication
(10) Pub. No.: US 2004/0241195 A1
(43) Pub. Date: Dec. 2, 2004

(54) Volatile material-containing compositions

(75) Inventor: Fernando Ray Tollens, Indian Hills, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/447,749

(22) Filed: May 29, 2003

(51) Int. Cl. A61K 7/46; A61L 9/015; A01N 25/00

(52) U.S. Cl. 424/401; 424/76.2; 424/405; 512/1

(57) ABSTRACT

Volatile material-containing compositions; methods of making volatile material-containing compositions; and methods of emitting a volatile material from a volatile material-containing composition are disclosed.

### FIELD OF THE INVENTION

The present invention relates to volatile material-containing compositions; methods of making volatile material-containing compositions; and methods of emitting a volatile material from a volatile material-containing composition.

### BACKGROUND OF THE INVENTION

Volatile material-containing compositions are used for various purposes. Such purposes include, but are not limited to releasing into a room or other space, volatile materials such as perfumes or scented materials, insecticides, air fresheners, deodorants, aromacology, aromatherapy, or any other odor that acts to condition, modify, or otherwise charge the atmosphere or to modify the environment.

There are a number of drawbacks to known compositions used for these purposes. One primary drawback is the fact that most of such compositions will allow the volatile materials to volatilize and escape, even when the volatile material-containing composition is not intended to be in use, nor to be emitting volatile materials therefrom. In the case of perfume-containing volatile compositions, this may also result in the composition releasing the more highly volatile perfume ingredients (referred to as the “top notes”) prematurely, and before the less volatile bottom notes and middle notes. This can result in an undesirable change in the scent character in some prior compositions over time, since the earlier emitted forms of the scents will comprise a disproportionately high proportion of top notes, and the later emitted forms of the scent, will comprise disproportionately higher proportions of the bottom notes. It is often desirable to emit the top, middle, and bottom notes together to provide a desired fragrance or scent. Another drawback, in the case of perfume-containing volatile compositions, is that the perfume ingredients will often not be compatible with the material of many containers.

### SUMMARY OF THE INVENTION

This invention relates to volatile material-containing compositions; methods of making volatile material-containing compositions; and methods of emitting a volatile material from a volatile material-containing composition. Several non-limiting embodiments are described herein, each of which may constitute an invention in its own right or together with other components.

In one non-limiting embodiment, the volatile material-containing composition comprises a structure that comprises: a carrier comprising at least one of the following: polyethylene glycol having a weight average molecular weight greater than or equal to about 4,000; hydrogenated castor oil; and fatty acids having a chain length greater than or equal to 14 carbon atoms; and at least one volatile material. In one version of this embodiment, the composition is in the form of a pliable solid at 25° C. and 50% relative humidity (RH). In certain embodiments, the volatile material(s) will only be released in limited amounts, if at all, until energy is applied to the structure in order to release the volatile material(s).

Methods of making volatile material-containing compositions; and methods of emitting a volatile material from a volatile material-containing composition are also disclosed.

Numerous other embodiments are also possible, including, but not limited to those described in the following detailed description.

### DETAILED DESCRIPTION OF THE INVENTION

This invention relates to volatile material-containing compositions; methods of making volatile material-containing compositions; and methods of emitting a volatile material from a volatile material-containing composition. Several non-limiting embodiments are described herein, as are several components of the system, each of which may constitute an invention in its own right or together with other components.

The volatile materials can be emitted in various facilities, which include but are not limited to rooms, houses, hospitals, offices, theaters, buildings, and the like, or into various vehicles such as trains, subways, automobiles, airplanes and the like.

The term “volatile materials” as used herein, refers to a material that is vaporizable. The terms “volatile materials”, “aroma”, and “scents”, as used herein, include, but are not limited to pleasant or savory smells, and, thus, also encompass scents that function as insecticides, air fresheners, deodorants, aromacology, aromatherapy, or any other odor that acts to condition, modify, or otherwise charge the atmosphere or to modify the environment. It should be understood, however, that perfumes, aromatic materials, and scents will often be comprised of one or more volatile materials (which may form a unique and/or discrete unit comprised of a collection of volatile materials).

In one embodiment, a system for dispensing scents into the environment can be provided which comprises one or more components containing one or more scents or aromatic materials. In such an embodiment, the system preferably comprises a dispensing device, such as a device and one or more aromatic material-containing articles of manufacture, or “scent-containing articles of manufacture”, which may be provided in the form of fragrance “cartridges”. Each cartridge can provide a single volatile composition, or a combination of different volatile materials, such as a combination of different scented materials. In certain embodiments, each of the cartridges provides a collection of scents that conveys, e.g., a theme, an experience, a physiological effect, and/or a therapeutic effect.

The volatile compositions of interest herein can be provided in any suitable form. In some embodiments, scents are provided by volatile compositions comprising perfume, such as perfume oils, that are incorporated onto or into a suitable carrier. The carriers can be provided in the following non-limiting forms: a solid, a liquid, a paste, a gel, beads, encapsulates, wicks, a carrier material, such as a porous material impregnated with or containing the perfume, and combinations thereof. In some embodiments, the carrier is in the form of a pliable solid which can be melted and have the perfume ingredients added thereto in order to form a composition that is in the form of a pliable solid structure or matrix at room temperature (73° F. (25° C.), 50% RH).

In certain embodiments, the volatile composition has a viscosity of from about 1,000 Cps to about 1,000,000 Cps, or more, measured at a shear stress of 100 Pa in a rotational rheometer, like the AR2000 (TA instruments New Castle, Del., USA), using a 40-mm diameter cone-and-plate geometry at 25 ° C. Such a composition can exist as a gel up to at least about 13,000 Cps. In certain embodiments when the composition is in the form of a pliable solid, it can have a viscosity of from about 100,000 to about 1,000,000 Cps.

In one non-limiting embodiment, at room temperature, the composition is in the form of a structure that is a structured polymeric pliable solid. Such a structure may be porous or non-porous. The structure may be homogeneous (which may also be referred to herein as “continuous”), or non-homogeneous. In many embodiments, it is desirable for the structure to be permeable to volatile materials contained therein. This will allow the structure to release the volatile materials contained therein when desired. In preferred versions of such an embodiment, the composition comprises a non-porous, homogeneous, permeable, structured polymeric pliable solid.

The volatile composition can be formed in a number of different manners. In one embodiment, the composition can be made by adding the volatile ingredient(s) to a carrier, such as polyethylene glycol (or “PEG”). The volatile ingredients, such as perfumes, are preferably miscible with the carrier, and after cooling, forms a pliable solid-like at room temperature. PEG is available in various molecular weights. While PEG's having low molecular weights (or “MW”) (e.g., molecular weights less than 400) can be used as solvents for perfumes, such PEG's are liquids at room temperature, and may be used, but are not preferred for use in the compositions described herein. In more preferred embodiments of the composition, the MW of PEG is greater than or equal to about 1,000, or greater than or equal to about 4,000. It is desirable that the MW of PEG be greater than or equal to about 8,000. The molecular weight of PEG may be as high as 24,000, or higher. All molecular weights specified herein are weight average molecular weights.

Other suitable carriers are hydrogenated castor oil and high chain fatty acids, particularly those with a chain length of greater than or equal to 14 carbon atoms. In certain embodiments, it is desirable for the majority of the composition to comprise such a carrier and the volatile ingredient(s). Thus, such a carrier and the volatile ingredient(s) may comprise more than about 20%, alternatively, more than about 50% of the composition, by weight. In certain embodiments, it may be desirable for the composition (and/or the carrier) to also be substantially free of HPC (hydroxy propyl cellulose).

It may be desirable to utilize a structurant with the carrier. A structurant can be used for any suitable purpose. Examples of such purposes include, but are not limited to providing the structure formed by the composition with greater stability. The structurant can reduce the tendency of the structure to release the volatile material(s) at low temperatures (e.g., ambient or storage or shipping temperatures). Thus, the volatile material(s) will not be released until energy is applied to the structure in order to release the volatile material(s). Any suitable structurant can be used. Suitable structurants comprise any substance that includes a divalent cation. Substances that comprise divalent cations include, but are not limited to magnesium and calcium containing molecules such as magnesium and calcium chloride, magnesium and calcium carbonate. Other suitable structurants include, but are not limited to derivatives of castor oil, including, but not limited to hydrogenated castor oil.

It may also be desirable for the composition to include at least one wax. Waxes can be used for any suitable purpose, including, but not limited to raising the melting temperature of structure formed by the composition for improved stability. Any suitable wax(es) can be used. In certain embodiments, it is desirable for the wax to have a melting point that is greater than that of the carrier. If the carrier is PEG, the melting point of the wax may, for example, be greater than about 50° C. Suitable waxes include, but are not limited to waxes that are derivatives of the carrier, for example, derivatives of PEG. Waxes that are derivatives of the carrier may be preferred because the structurants that are capable of structuring the carrier will also be able to structure the waxes in order to further raise the melting point of the entire matrix. It may also be desirable that the wax does not have an affinity for the volatile material so that it does not affect the emission rate or delivery of the volatile material.

In one embodiment, the composition is formed by combining polyethylene glycol (or “PEG”), hydrogenated castor oil, and a low level of at least one wax, and at least one volatile ingredient.

The volatile ingredient(s) can comprise a number of components or compositions, including, but not limited to: fragrances (or perfume oils), flavors, pesticides, repellants, or mixtures thereof.

The volatile ingredient(s) can be combined with the carrier material in any suitable manner. Several suitable manners in which the volatile ingredient(s) can be combined with the carrier material include, but are not limited to: by entrapment; the volatile ingredient(s) can be dissolved in the carrier material; the volatile ingredient(s) can be partially encapsulated or completely encapsulated in the carrier material.

The components of the composition can be incorporated into the composition in any suitable amounts. In some embodiments, it may be desirable for the concentration of the volatile material(s) to be greater than about 10% of the composition. In some embodiments, the concentration of the volatile material(s), such as the perfume ingredients, may be as high as about 75%, or more of the composition. In other embodiments, the amount of volatile material(s) may range range from about 25% to about 75% of the composition. The carrier (such as polyethylene glycol) may comprise the balance of the composition. In some embodiments, the carrier may range from about 25% to about 75%, or more. In alternative embodiments, the carrier may be present in an amount that is less than this range. The structurant (such as hydrogenated castor oil) level may range from about 0 to about 15%, 20%, 30%, 40%, or more. The wax level may range from about 0 to about 3%, 5%, or more. All percentages stated herein are by weight of the composition, unless stated otherwise. The amounts of the components are typically selected so that they total 100%. However, it is also possible for other components to be added to the composition, in which case the weights of the components such as the carrier, volatile material(s), structurant, and wax may total less than 100% of the composition.

The composition can be made in any suitable manner. In one non-limiting embodiment, the composition is formed by heating the carrier material (such as PEG) until it melts, and adding the volatile material(s) to the molten PEG. If the carrier is PEG, it will typically melt at about 100° C. to about 120° C. The PEG serves as a solvent for perfume oils, and perfume oils are completely miscible in the same. Addition of the volatile material(s) will quench the PEG to a lower temperature and form a pliable solid when it cools to room temperature. In embodiments in which a structurant is used, the carrier material and the structurant may be mixed and heated so that they melt together. The volatile material(s) can then be added to the molten mixture of the carrier and structurant. In embodiments in which a wax is used, the carrier material, the structurant, and the wax are mixed and heated so that they melt together. The volatile material(s) can then be added to the molten mixture of the carrier, structurant, and wax.

The structure (or matrix) comprising the composition can be thermally triggered or otherwise energized to emit the volatile material(s). Such a structure can undergo a transition between a variety of different states depending on the temperature to which the structure is heated. For instance, in some embodiments, the composition can exist in any of the following phases: solid, paste, gel, semi-molten, and liquid, or other states. Each phase of the composition can provide different volatilization characteristics. In the case of scented materials, this can include different volatilization rates, intensities, scent characters, emission profiles, etc. In some embodiments, the change in state of the composition is reversible in that it can change back to, or toward, more solid states. In some embodiments, it may be possible to vary the form or state of the composition from solid-like to gel-like by controlling the proportions of the components of the composition. For example, the composition will become less solid-like and more gel-like with the addition of additional structurant, such as hydrogenated castor oil. The reversible liquefication/gellation/solidification of the structure can be used to regulate/control the release of the volatile material. In most compositions, in the case of fragrance compositions, at lower temperatures, the more highly volatile perfume components (the “top notes”) will volatilize first. In the case of certain embodiments of the compositions described herein, if the composition is heated above its melting point (until it becomes a liquid), the perception of the volatile composition will be more true to the desired essence of the character, scent, flavor, etc. of the volatile material since all of the components of the material will be emitted at the same intensity at the desired temperature and time from the highly volatile perfume components (the “top notes”) to the less volatile (“bottom notes”). Thus, in certain embodiments, there is minimum partitioning of the volatile material composition and consistency of character/concentration over time. In the case of the examples set out herein, the melting point of the matrix is about 52° C. When energy is no longer applied, the structure goes back to a wax-like solid state or pliable solid which reduces the tendency of the volatile material to escape.

In certain embodiments, it is desirable for the composition to be heated to a temperature that is in excess of the melting point of the carrier. The addition of perfume ingredients will typically lower the melting temperature of the composition. As perfume ingredients are volatilized, the melting temperature of the remaining portion of the composition will increase. If the composition is always heated to a melting temperature above that of the carrier, then this will always provide sufficient energy to the composition in order to emit the volatile components therefrom.

The composition may provide certain advantages. It should be understood in this regard, however, that the composition need not provide any of these advantages unless specified in the appended claims. In some embodiments in the case of fragrance compositions, the composition can deliver a longer lasting aroma. For example, certain gels which have been previously used to contain volatile materials will release the more volatile perfume components even without being heated, or otherwise energized. This will reduce the longevity of such compositions, and will effect the character of the perfume that is emitted when the composition is heated. In some embodiments, the composition can retain the volatile material(s) better than some other compositions during periods when the volatile material(s) are not intended to be emitted. In some embodiments, the composition can be more compatible with the material of the container in which is placed (which may be referred to as “supporting material”). Often perfume oils are not compatible with plastics. However, when perfume oils are incorporated into the composition described herein, the composition may be more compatible with plastic materials. Without wishing to be bound to any particular theory, it is believed that the volatile material-containing composition described herein will have a greater surface tension than that of the perfume oil, to reduce or eliminate migration of the perfume oil from the composition, a phenomenom known as wicking. In some embodiments, the composition will have a surface tension of higher than 20 dyne/cm and lower than 25 dyne/cm. In some embodiments, the composition will have good stability at elevated temperatures (e.g., up to about 120° F., or 50° C.) and/or high humidity (e.g., up to; or greater than or equal to about 80% RH), even at high volatile material concentrations. That is, the composition will not change shape or physical state under such conditions. In certain embodiments, the composition provides a structure that will not change its physical state (e.g., become more liquid) even when it absorbs water, such as humidity.

The composition may, in some embodiments, also be advantageous in that it may contain relatively high levels of volatile material (e.g., from about 25% to about 75% by weight of the composition). The composition can also incorporate a large number, range, spectrum (or portfolio) of different volatile materials. This is possible due to the ability to alter/adjust the polarity of the carrier to match the polarity of the volatile material by modifying the level of the structurant (e.g., hydrogenated castor oil). For example, in the case of the compositions described herein, the polarity of the volatile material(s) can be in the range of from about 2 to about 5 Debyes, yet the compositions may still be stable under a wide range of storage conditions. This allows combinations of perfumes that are typically not compatible to be incorporated into compositions (for example, vanilla, coffee, cinnamon, which are very polar, can be combined with fruits (e.g., lemon), or other types of perfume ingredients that are at the other end of the polarity spectrum. In addition, the structure of the composition that incorporates the volatile material(s) may be reversible (that is, it can be converted from a more solid state (e.g, a pliable solid) to a more liquid state, and then back to a more solid state). This may provide the composition with handling, storing, and processability benefits.

### EXAMPLES

Table 1 provides some non-limiting examples of scented compositions that can be made according to the description herein.

TABLE 1

| | | Hydrogenated | |
|---|---|---|---|
| Perfume % | PEG % | Castor Oil % | Wax % |
| 25 | 75 | 0 | 0 |
| 50 | 45 | 5 | 0 |
| 40 | 30 | 30 | 0 |
| 40 | 40 | 20 | 0 |
| 40 | 48 | 10 | 2 |
| 50 | 42 | 5 | 3 |
| 25 | 0 | 75 | 0 |

The disclosure of all patents, patent applications (and any patents which issue thereon, as well as any corresponding published foreign patent applications), and publications mentioned throughout this description are hereby incorporated by reference herein. It is expressly not admitted, however, that any of the documents incorporated by reference herein teach or disclose the present invention.

It should be understood that every maximum numerical limitation given throughout this specification will include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

While particular embodiments of the subject invention have been described, it will be obvious to those skilled in the art that various changes and modifications of the subject invention can be made without departing from the spirit and scope of the invention. In addition, while the present invention has been described in connection with certain specific embodiments thereof, it is to be understood that this is by way of illustration and not by way of limitation and the scope of the invention is defined by the appended claims which should be construed as broadly as the prior art will permit.

What is claimed is:

1. A volatile material-containing composition comprising:
a carrier comprising at least one of the following: polyethylene glycol having a weight average molecular weight greater than or equal to about 4,000; hydrogenated castor oil;
and fatty acids having a chain length greater than or equal to 14 carbon atoms; and
at least one volatile material,
wherein said composition is in the form of a pliable solid at 25° C. and 50% RH.

2. A volatile material-containing composition according to claim 1 wherein said composition has a viscosity between about 100,000 and 1,000,000 Cps.

3. A volatile material-containing composition according to claim 1 wherein said composition remains a pliable solid under at least one of the following conditions: at temperatures up to about 50° C., and at a humidity greater than or equal to about 80% RH.

4. A volatile material-containing composition according to claim 1 wherein said at least one volatile material comprises one or more of the following: perfume oils, flavors, pesticides, repellants, and mixtures thereof.

5. A volatile material-containing composition according to claim 4 wherein said composition contains up to about 75% of volatile material by weight.

6. A volatile material-containing composition according to claim 2 wherein said the pliable solid retains at least about 80% of said volatile material at temperatures up to about 50° C. when completely exposed for about 2 weeks.

7. A volatile material-containing composition according to claim 5 wherein said composition can be thermally triggered at temperatures above 50° C. to emit at least some of the volatile material.

8. A volatile material-containing composition according to claim 1 further comprising a structurant.

9. The volatile material-containing composition of claim 8 wherein the structurant is a substance that comprises a divalent cation.

10. The volatile material-containing composition of claim 9 wherein the structurant is selected from the group consisting of: magnesium and calcium containing molecules such as magnesium and calcium chloride, magnesium and calcium carbonate, and mixtures thereof.

11. The volatile material-containing composition of claim 9 wherein said structurant comprises a derivative of castor oil.

12. The volatile material-containing composition of claim 1 further comprising at least one wax.

13. The volatile material-containing composition of claim 12 wherein said carrier has a melting temperature and said wax has a melting temperature that is higher than the melting temperature of said carrier.

14. The volatile material-containing composition of claim 12 wherein said wax is a derivative of said carrier material.

15. The volatile material-containing composition according to claim 1 wherein said composition has a first state when said composition is in the form of a pliable solid and energy is not applied to said composition, and a second energized state when energy is applied to said composition, wherein the volatile material is emitted at a first level from said volatile material-containing composition in said first state and the volatile material is emitted from said volatile material-containing composition at a second higher level in said second state; wherein said volatile material-containing composition returns to said first state when energy is no longer applied to said volatile material-containing composition.

16. The volatile material-containing composition according to claim 15 wherein said composition is melted in said second state.

17. The volatile material-containing composition according to claim 1 wherein said volatile material has a surface tension, and said volatile material-containing composition is placed on a supporting material, and the volatile material-containing composition has a surface tension that is greater than that of the volatile material alone, and the surface tension of the volatile material-containing composition is higher than 20 dyne/cm and lower than 25 dyne/cm.

18. A volatile material-containing composition comprising:
polyethylene glycol comprising between about 25% and about 75%, by weight of said composition; and
at least one volatile material, comprising between about 10% and about 75%, by weight of said composition; and
optionally other ingredients,
wherein said composition is in the form of a pliable solid at room temperature.

19. A method of making a volatile material-containing composition, said method comprising:
providing a carrier material
heating said carrier material until it melts;
adding at least one volatile material to the molten carrier material to form a mixture; and
allowing the mixture of said molten carrier material and said at least one volatile material to cool.

20. A method of emitting a volatile material from a volatile material-containing composition, said method comprising:
providing a volatile material-containing composition comprising a carrier and at least one volatile material that is miscible in said carrier, wherein said carrier has a melting temperature, and said composition has a melting temperature that is lower than the melting temperature of said carrier, wherein said composition has a first state when energy is not applied to said composition, and a second energized state when energy is applied to said composition,; and
heating said volatile material-containing composition to a temperature above that of the melting temperature of said carrier.

21. The method of claim 20 wherein when heat is no longer applied to said volatile material-containing composition, said composition returns to said first state.

22. The method of claim 21 wherein the first state of said composition is a pliable solid.
